(19) United States
(12) Patent Application Publication
(10) Pub. No.: US 2022/0351857 A1
(43) Pub. Date: Nov. 3, 2022

(54) PREDICTIVE CHANGE IN ACUITY FOR HEALTHCARE ENVIRONMENTS

(71) Applicant: Galerie Technology, LLC, Atlanta, GA (US)

(72) Inventors: Julia Yudkovicz, Atlanta, GA (US); Rob Lewis, Atlanta, GA (US); Timothy T. Gary, Atlanta, GA (US); David Harold Frakes, Rancho Santa Fe, CA (US)

(21) Appl. No.: 17/733,271

(22) Filed: Apr. 29, 2022

Related U.S. Application Data
(60) Provisional application No. 63/181,388, filed on Apr. 29, 2021.

(51) Int. Cl. G16H 50/20; G16H 20/70; G16H 40/63; G16H 40/20; G16H 20/30

(52) U.S. Cl. CPC G16H 50/20; G16H 20/70; G16H 40/63; G16H 40/20; G16H 20/30

(57) ABSTRACT

Disclosed are various embodiments for implementing predictive change in acuity. A computing environment may be employed to store behavioral data received from a plurality of monitoring devices associated with individuals. The computing environment may generate training data using the behavioral data, train a predictive algorithm using the training data, and execute at least one predictive data model as trained using subsequent behavioral data as received from the plurality of monitoring devices to predict at least one of a change in behavior of one of the individuals; an incident associated with the one of the individuals; and a change in biometric statistics of the one of the individuals.

### CROSS-REFERENCE TO RELATED APPLICATION

This application claims priority to co-pending U.S. provisional application entitled, “Predictive Change in Acuity for Healthcare Environments,” having Ser. No. 63/181,388, filed Apr. 29, 2021, which is entirely incorporated herein by reference.

### BACKGROUND

Today, assisted living facilities require personnel to constantly interact with residents to ensure the health, happiness, and general well-being of its residents. Slight changes to acuity, behavior, and/or medical conditions cannot always be observed and/or predicted by personnel despite the constant interactions and residents may be less than forthcoming. In this case, acuity refers to the level of care appropriate for a resident. For example, high-acuity care has been defined as an individualized solution that honors care preferences and avoids costly, unnecessary interventions.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of an example spider diagram for generating a behavioral score according to various embodiments of the present disclosure.

FIG. 2 is a drawing of an example spider diagram for generating a behavioral score according to various embodiments of the present disclosure.

FIGS. 3A-3B show examples of a 4-point chart representation for generating a behavioral score pattern according to various embodiments of the present disclosure.

FIG. 4 is a schematic block diagram that provides one example illustration of a computing environment employed in a networked environment according to various embodiments of the present disclosure.

### DETAILED DESCRIPTION

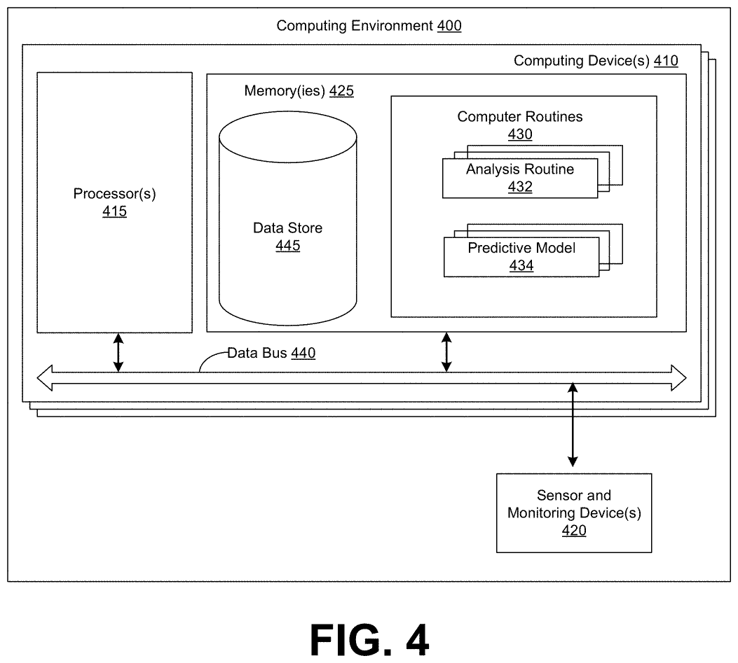

The present disclosure relates to predictive change in acuity for healthcare environments. In some embodiments, resident behaviors, such as activities-of-daily-life (ADLs) and information-of-things (IoT) sensor information of individuals are collected and recorded in a computing environment 400 (FIG. 4) by sensor & monitoring devices 420 (FIG. 4) (e.g., motion sensors, heartrate sensors, toilet flush sensors, wearable sensors, video cameras, etc.) for use in a healthcare setting. For instance, the healthcare setting may include a hospital, an assisted care facility, an assisted living facility, or other healthcare-related facility.

A computing environment 400 (FIG. 4) comprising at least one or more computing devices 410 may be employed to correlate resident behaviors (as monitored by sensor & monitoring device(s) 420) with events to better understand cause and effect, allowing staff and family to proactively intercede and provide a better resident experience, as well as predict changes in resident acuity more accurately, providing greater opportunities for preventative care. To achieve the foregoing, various embodiments are described herein to collect and store data, compile resident daily data profiles, and leverage computer routines 430 (FIG. 4), which can include predictive algorithms and machine learning (ML) technologies to analyze and interpret patient information based on observed data. In some embodiments, the computing environment 400 (FIG. 4) does not diagnose causality, but observes correlations and trend deviations. An iterative approach to data modeling may be employed where a reasonably limited number of variables are investigated first, evaluated for usefulness, and modified until desired results are achieved.

Data Collection. Initially, behaviors may be manually recorded and stored in a data store 445 (FIG. 4) (e.g., memory 425 (FIG. 4)) of the computing environment 400 (FIG. 4) to serve as training set across a population of individuals. As caregivers or other personnel assist with ADLs, various behaviors may be recorded. In some embodiments, the behaviors include behavioral metrics, such as a calm metric, an agreeable metric, a sociable metric, a confused metric, a combative metric, an agitated metric, alertness metric, mood metric, sleep metric, activity metric, pain metric, medication (meds) metric, gait metric, etc. although other metrics may be employed and a subset of the above metrics may be employed.

In some embodiments, the data may be initially evaluated after a predetermined number of observations (e.g., 1000 observations) or after a predetermined amount of time (e.g., three months). The data may then be reviewed to generate reliable data models and/or computer routines, such as predictive data models 434, logistic regression models, etc. Data collection may be monitored to ensure proper and consistent methodology is employed.

In some embodiments, the computing environment 400 (FIG. 4) may incorporate IoT sensor information into a resident daily data profile. An objective is to build a thorough time-series of resident activity that can later be correlated with behaviors, incidents (falls), and monitored activities (e.g., sleep quality, activity levels, etc.). A community operating platform comprising a series of one or more user interfaces may be provided by the computing environment 400 (FIG. 4) to streamline collection of data, improve accuracy, and increase volume of data. The user interface(s) may be accessible by at least one of a family user account, a physician user account, and a staff member user account

Behavioral Correlation. In some embodiments, the computing environment 400 (FIG. 4) may leverage association rule learning techniques to uncover correlations between behaviors and data collected in the resident daily data profile by sensor & monitoring device(s) 420 (FIG. 4). For instance, “Mrs. Smith is always combative with Caregiver Sam,” and “Mr. Jones is always confused at breakfast,” are example behaviors.

Modeling. In some embodiments, the computing environment 400 (FIG. 4) may leverage supervised outlier detection techniques to identify anomalies as part of preprocessing, such as “Does a resident suddenly move from agreeable to combative?,” and “Does a resident suddenly show a drastic reduction in motion within their unit?” If an anomaly is identified, the computing environment 400 (FIG. 4) may determine whether the event appears legitimate or should it be removed from the training set. In some embodiments, manual inspection may be employed to determine whether an event is legitimate and/or should be removed from a training set. This inspection may be applied across the population first followed by individuals. Outlier detection routines may be executed to identify valuable information about a population and individuals in a fairly short time horizon

Once outliers are verified or scrubbed, the computing environment 400 (FIG. 4) may execute one or more machine learning routines (e.g., regression or other suitable options) to model observations with likely outcomes, such as “Mrs. Smith has 20% more ‘confused’ observations and 32% fewer toilet flushes over the past three days, so she may be trending toward a urinary tract infection (UTI).” As may be appreciated, accurate predictive models 434 (FIG. 4) require a large volume of data (and therefore a long collection timeline) for precision. In order to produce the predictive data models, training data is generated using behavioral data and is used to train a predictive algorithm, such that a predictive data model can be executed using subsequent behavioral data to predict a health change attributed to a monitored behavior, a medical event, and/or a change in biometric statistics of an individual.

The computing environment 400 (FIG. 4) may compare results to existing data models for medical risk prediction to evaluate accuracy, such as ADL/IADL; DXCG; CCI; CMS-HCC (hierarchical condition codes); any combination thereof; and other models.

Additional Measures—Resident Score. In some embodiments, the computing environment 400 (FIG. 4) may employ the aforementioned information to generate and output a resident score, which may include, for example, a composite of ADL completion, behaviors, monitored activities, incidents, and/or observed mobility (sensors). In some embodiments, the resident score may be computed by a computing device 410 based on monitored observations via sensor & monitoring device(s) and/or the score may be inputted based on reports from the subject resident or healthcare providers and staff. In some embodiments, the resident score may be presented to family, physicians, staff, or other individuals. If suitable permissions are held, the computing environment 400 (FIG. 4) may progressively disclose more information. To this end, in some embodiments, each resident will naturally have a different score and every resident score will naturally degrade with time as they age. A baseline score may be established, for example, that will likely move lower over time, for each resident and highlight when they move significantly from that baseline. When a deviation beyond a threshold is detected, a warning signal or a warning event may be generated and/or a corrective action may be initiated, such as escalating the warning event to a supervisor or medical personnel for evaluation via prompt communication messaging or calling. A weighted ensemble of models may be employed to achieve the foregoing, which may include multiple underlying models that are combined into a single model.

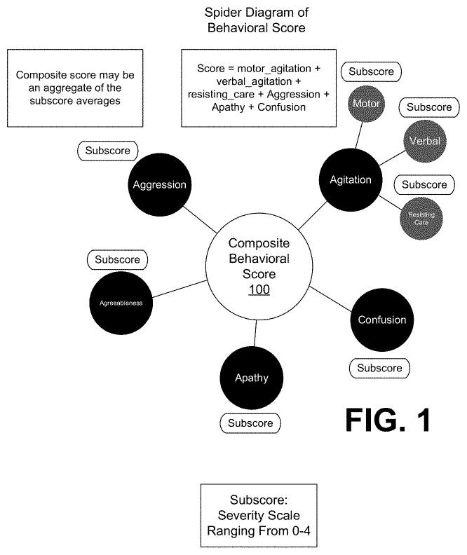

Referring now to FIG. 1, an overall behavioral score 100, also referring to as a resident score, is shown that may include a composite of six distinct sub-scores that align with different types of behaviors. The diagram of FIG. 1 shows the constituents of the composite score as well how the constituents may be scored. Each distinct behavior may be scored via an analysis routine 432 (FIG. 4), for instance, on a scale of 0-4, with values increasing in accordance with the perceived severity of the behavior being recorded. Possible values for the composite score will range from 0-24, with lower scores indicating minimal presence of disruptive, negative behaviors, whilst higher scores indicate that a resident is displaying severe, negative behaviors that may be disruptive to the staff or other residents. Other scoring concepts may be implemented, as may be appreciated. The composite score may be utilized as an alert mechanism for staff, as opposed to a predictor in future statistical analyses.

Analysis Routine. However, the individual sub-scores obtained from the distinct behaviors (e.g., a calm demeanor, agreeability, clear-headedness, sociability, aggression, apathy, confusion, motor agitation, verbal agitation, resisting care, alertness, mood, sleep, activity, pain, medication (meds) levels, gait, etc.) may be used via an analysis routine 432 to generate a prediction model 434 for an individual resident. The computing environment 400 (FIG. 4) may predict a health change attributed to a monitored behavior of one of the individuals; a medical event with the one of the individuals; and a change in biometric statistics of the one of the individuals. Thus, the computing environment 400 (FIG. 4) may estimate the risk or the probability of a certain outcome or medical event, such as the event of a stroke, while accounting for additional covariates such as frailty status, cognitive function, vascular health (as indicated by systolic blood pressure, pulse, etc.), and medical history.

Logistic Regression. In order to determine which distinct behaviors and covariates are capable of predicting a specific outcome, a logistic regression model may be generated and employed using a portion of the data, which is known as the training dataset. This method may be used by the computing environment 400 (FIG. 4) to determine which covariates are significantly associated with either a greater or lower odds of experiencing a specific outcome (e.g., a stroke). The significant predictors may also indicate which biological and behavioral factors are causally associated with an outcome variable.

Conditional Probability Tables (CPTs). Based on the variables which are significantly associated with the odds of a specific outcome, a conditional probability table may be constructed in order to later visualize the conditional probability distribution with respect to discrete variables.

Decision Graphs. Utilizing the conditional probability tables, decision graphs will be constructed for each unique outcome variable to be evaluated.

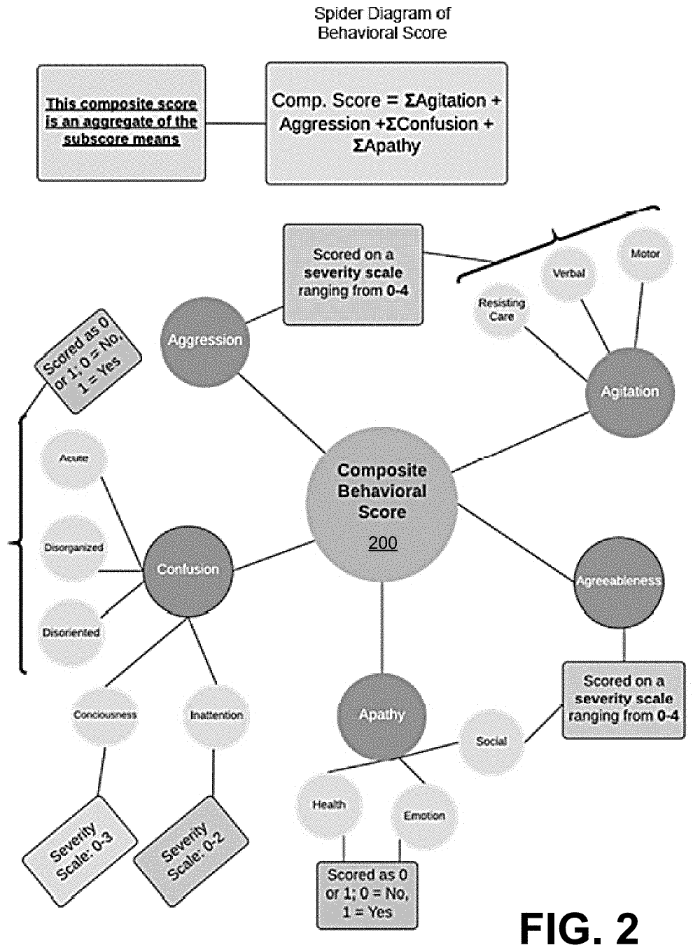

Establishing Behavioral Score. Referring now to FIG. 2, in some embodiments, an overall behavioral score 200 may be generated by an analysis routine 432 as a function of six distinct sub-scores that align with different types of behaviors. FIG. 2 describes the constituents of the composite score as well how the constituents will be scored.

For instance, distinct behaviors may be scored on either a severity scale or on the basis of whether this behavior is present or not. Possible values for the composite score may range from 0-26, with lower scores indicating minimal presence of disruptive, negative behaviors, whilst higher scores indicate that a resident is displaying severe, negative behaviors that may be disruptive to the staff or other residents. In some embodiments, these composites scores may be provided as a weighted estimate of general behavioral well-being (weighted by level of assistance required or ADLs, for example). The composite score may be utilized as an alert mechanism for staff, as it is not likely to be used as a predictor in future statistical analyses.

Analysis Plan. However, the individual sub-scores obtained from the distinct behaviors (e.g., a calm demeanor, agreeability, clear-headedness, sociability, aggression, apathy, confusion, motor agitation, verbal agitation, resisting care, alertness, mood, sleep, activity, pain, medication (meds) levels, gait, etc.) may be used via an analysis routine 432 to generate a prediction model 434 for an individual resident. Thus, utilizing the sub-scores obtained from the distinct behaviors, it is desirable to estimate the risk or the probability of a certain outcome such as the event of a stroke, while accounting for additional covariates such as frailty status, cognitive function, vascular health (as indicated by systolic blood pressure, pulse, etc.), and medical history.

Patterns and Associations. K-nearest neighbors and Principal Component Analysis. Both of these methods are unsupervised machine learning routine that may be implemented in order to initially identify which baseline characteristics may be associated with each other as well as with outcome measures. Using graphs, an analysis routine 432 can measure the distance between participants on an x-y plane in order to determine the degree of similarity among residents, based on a particular grouping feature (i.e., which feature or measure results in greater discernibility between the participants). It is understood that the present disclosure is not limited to K-nearest neighbors and principal component analysis, and other pattern and association models may be employed.

Prediction Models. A random forest is a supervised machine learning method, which outputs the probability of a participant developing a certain outcome. It is composed of hundreds of decision trees, each representing a unique medley of variables and subsamples, otherwise known as a bootstrapped dataset. In various embodiments, a bootstrapped dataset is created by randomly selecting a subsample of participants and subset of variables, which are used to compose a decision tree. The innate variety in exemplary trees allows random forests to be more flexible when it comes to classifying new samples, thus improving accuracy. It is understood that the present disclosure is not limited to random forests, and other prediction models may be employed.

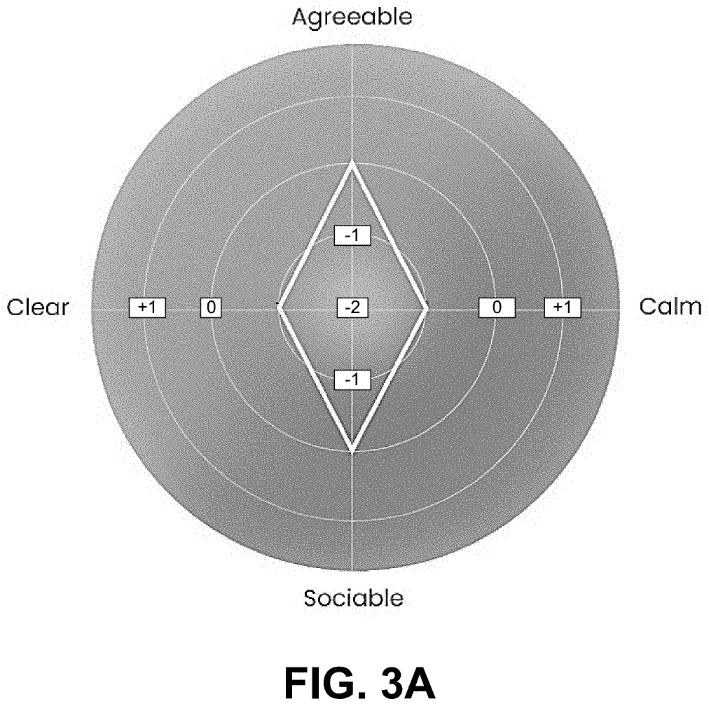

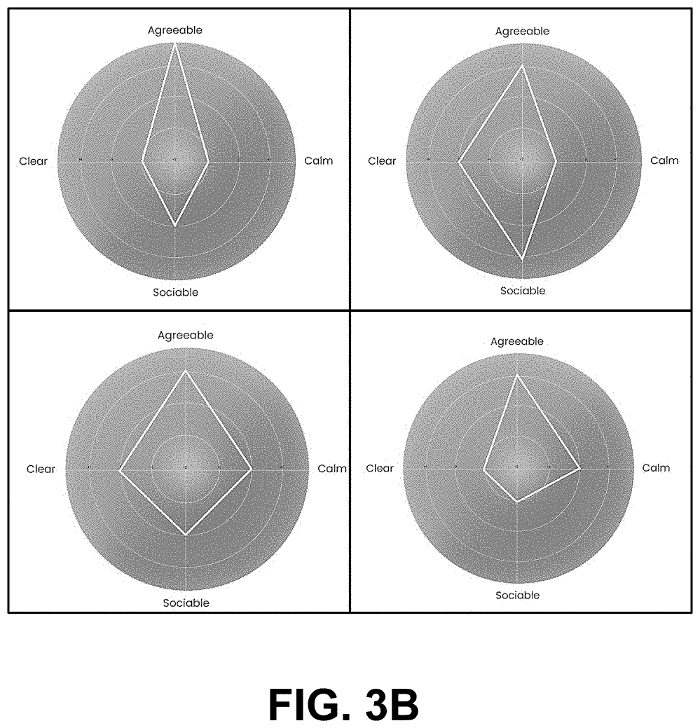

With reference to FIG. 3A, shown is a 4-point chart representation of 4 distinct sub-scores that align with different types of behaviors. The diagram of FIG. 3A shows four sub-scores for four types of behaviors: clear (thinking or headedness), sociability, agreeability, and calm(ness). In the example of FIG. 3A, the resident has received scores of −1 for clear and calm and scores of 0 for agreeable and sociable. Based on the computed scores, lines can be drawn between respective scores to form a pattern, such as a diamond pattern represented in FIG. 3A. Accordingly, different patterns can result from different scores, as shown in FIG. 3B, where 4 different charts are provided illustrating 4 different patterns representing various combinations of individual behavior scores. Additionally, over time, behavior patterns of a resident may change in progression as the resident moves between different behavior states. Thus, charts of a resident may be compared with earlier charts of the resident to predict future changes in behavior, an incident, and/or biometric statistics that correlate with the progression of pattern changes for the resident.

With reference to FIG. 4, shown is a schematic block diagram of the computing environment 400 according to an embodiment of the present disclosure. The computing environment 400 includes one or more computing devices 410. Each computing device 410 includes at least one processor circuit 415, for example, having a processor 415 and a memory 425, both of which are coupled to a local interface 440. To this end, each computing device 410 may comprise, for example, at least one server computer or like device. The local interface 440 may comprise, for example, a data bus with an accompanying address/control bus or other bus structure as can be appreciated.

Stored in the memory 425 are both data and several components that are executable by the processor 415. In particular, stored in the memory 425 and executable by the processor 415 are the routines (e.g., analysis routine 432) described herein, and potentially other applications. Also stored in the memory 425 may be a data store 445 and other data, including conditional probability tables, decision graphs, etc. In addition, an operating system may be stored in the memory 425 and executable by the processor 415.

It is understood that there may be other applications that are stored in the memory 425 and are executable by the processor 415 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 425 and are executable by the processor 415. In this respect, the term “executable” means a program file that is in a form that can ultimately be run by the processor 415. Examples of executable programs may be, for example, a compiled program that can be translated into machine code in a format that can be loaded into a random access portion of the memory 425 and run by the processor 415, source code that may be expressed in proper format such as object code that is capable of being loaded into a random access portion of the memory 425 and executed by the processor 415, or source code that may be interpreted by another executable program to generate instructions in a random access portion of the memory 425 to be executed by the processor 415, etc. An executable program may be stored in any portion or component of the memory 425 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 425 is defined herein as including both volatile and nonvolatile memory and data storage components. Volatile components are those that do not retain data values upon loss of power. Nonvolatile components are those that retain data upon a loss of power. Thus, the memory 425 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 415 may represent multiple processors (e.g. graphics processing units, central processing units, etc.) and/or multiple processor cores and the memory 425 may represent multiple memories that operate in parallel processing circuits, respectively. In such a case, the local interface 440 may be an appropriate network that facilitates communication between any two of the multiple processors, between any processor and any of the memories, or between any two of the memories, etc. The local interface 440 may comprise additional systems designed to coordinate this communication, including, for example, performing load balancing. The processor 415 may be of electrical or of some other available construction.

Although various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein that comprises software or code can be embodied in any non-transitory computer-readable medium for use by or in connection with an instruction execution system such as, for example, a processor 415 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein may be implemented and structured in a variety of ways. For example, one or more applications described may be implemented as modules or components of a single application. Further, one or more applications described herein may be executed in shared or separate computing devices 410 or a combination thereof. For example, a plurality of the applications described herein may execute in the same computing device 410, or in multiple computing devices in the same computing environment 400. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A system for predictive change in acuity, comprising:
at least one computing device; and
program instructions stored in memory and executable in the at least one computing device that, when executed, direct the at least one computing device to:
store behavioral data received from a plurality of monitoring devices associated with individuals;
generate training data using the behavioral data;
train a predictive algorithm using the training data; and
execute at least one predictive data model as trained using subsequent behavioral data as received from the plurality of monitoring devices to predict at least one of: a health change attributed to a monitored behavior of one of the individuals; a medical event with the one of the individuals; and a change in biometric statistics of the one of the individuals.

2. The system of claim 1, wherein the behavioral data comprises a calm metric, an agreeable metric, a clear metric, or a sociable metric.

3. The system of claim 1, wherein the plurality of monitoring devices comprise a wearable device having at least one sensor configured to measure biometric information of an individual or interaction with a feature of a healthcare facility.

4. The system of claim 3, wherein the interaction with a feature of the healthcare facility comprises an interaction with a toilet, a shower, or other fixture of the healthcare facility.

5. The system of claim 1, wherein the at least one computing device is further directed to:
generate and store a daily data profile for an individual in the memory; and
generate a relationship between behavioral data collected for the individual and data of the daily data profile generated for the individual.

6. The system of claim 1, wherein the at least one computing device is configured to generate and store at least one of the following models: ADL/IADL; DXCG; CCI; and CMS-HCC (hierarchical condition codes).

7. The system of claim 1, wherein the at least one computing device is further directed to identify outliers in the behavioral data and remove the outliers from the behavioral data prior to identifying the training data from the behavioral data.

8. The system of claim 1, wherein the at least one computing device is further configured to execute a regression-based machine learning routine, the regression-based machine learning routine being configured to model observations and generate a likely outcome associated with one of the individuals.

9. The system of claim 1, wherein the at least one computing device is further directed to generate a resident score for each of the individuals based on at least one output of at least one machine learning routine.

10. The system of claim 9, wherein the resident score is generated as a function of an activities-of-daily-living (ADL) profile, a behavioral profile, current or past biometrics, past incident, and observed mobility.

11. The system of claim 10, wherein the at least one computing device is further directed to present the resident score in at least one user interface accessible by at least one of a family user account, a physician user account, and a staff member user account.

12. The system of claim 11, wherein the at least one computing device is further directed to present varying levels of information associated the resident score and/or the individual based on at least an account accessing the information being the family user account, the physician user account, and the staff member user account.

13. The system of claim 9, wherein each of the individuals has a different resident score, each of the resident scores changing with time as a corresponding individual ages.

14. The system of claim 10, wherein the at least one computing device is further directed to generate a baseline score that moves over time for each of the individuals and compare the resident score to the baseline score.

15. The system of claim 14, wherein the at least one computing device is further directed to generate a warning when the baseline score is beyond a predefined threshold of the resident score.

16. A method for predictive change in acuity, comprising:
storing, by at least one computing device, behavioral data received from a plurality of monitoring devices associated with individuals;
generating, by the at least one computing device, training data using the behavioral data;
training, by the at least one computing device, a predictive algorithm using the training data; and
executing, by the at least one computing device, at least one predictive data model as trained using subsequent behavioral data as received from the plurality of monitoring devices to predict at least one of: a health change attributed to a monitored behavior of one of the individuals; a medical event with the one of the individuals; and a change in biometric statistics of the one of the individuals.

17. The method of claim 16, wherein the plurality of monitoring devices comprise a wearable device having at least one sensor configured to measure biometric information of an individual or interaction with a feature of a healthcare facility.

18. The method of claim 16, further comprising:
generating and storing, by the at least one computing device, a daily data profile for an individual in memory; and
generating, by the at least one computing device, a relationship between behavioral data collected for the individual and data of the daily data profile generated for the individual.

19. The method of claim 16, further comprising:
generating, by the at least one computing device, a resident score for each of the individuals based on at least one output of at least one machine learning routine;
wherein each of the individuals has a different resident score, each of the resident scores changing with time as a corresponding individual ages;
generating, by the at least one computing device, a baseline score that moves over time for each of the individuals and comparing the resident score to the baseline score; and
generating, by the at least one computing device, a warning when the baseline score is beyond a predefined threshold of the resident score.

20. A non-transitory, computer-readable medium comprising machine-readable instructions that, when executed by a processor of a computing device, cause the computing device to at least:
store behavioral data received from a plurality of monitoring devices associated with individuals;
generate training data using the behavioral data;
train a predictive algorithm using the training data; and
execute at least one predictive data model as trained using subsequent behavioral data as received from the plurality of monitoring devices to predict at least one of: a health change attributed to a monitored behavior of one of the individuals; a medical event with the one of the individuals; and a change in biometric statistics of the one of the individuals.
